(19) United States
(12) Patent Application Publication
(10) Pub. No.: US 2007/0186933 A1
(43) Pub. Date: Aug. 16, 2007

(54) SYSTEMS AND METHODS FOR DELIVERING FLOW RESTRICTIVE ELEMENT TO AIRWAY IN LUNGS

(75) Inventors: Nicanor Domingo, Santa Clara, CA (US); Jeffrey Lee, San Lorenzo, CA (US); Anthony Wondka, Thousand Oaks, CA (US); George Surjan, San Jose, CA (US); Lutz Freitag, Hemer (DE)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PULMONx, Palo Alto, CA (US)

(21) Appl. No.: 11/624,136

(22) Filed: Jan. 17, 2007

Related U.S. Application Data
(60) Provisional application No. 60/759,713, filed on Jan. 17, 2006.

(51) Int. Cl. A61B 17/00; A61M 16/00

(52) U.S. Cl. 128/207.15; 606/206; 606/205

(57) ABSTRACT

A method and system for catheter-based delivery of implants in the body. Implants can include stents, plugs, coils, baskets, filters, valves, grafts, prosthesis', drugs, drug reservoirs, biologics, or pumps. The catheter system comprises a uniquely configured grasper mechanism that allows holding the implant during the unsheathing delivery step prior to full release. With this delivery system, the implant can be unsheathed, positioned, and the position can be evaluated prior to releasing the implant from the catheter. Upon evaluation of the position of the implant, if it is found to be inaccurately placed, then removal of the implant can be done easily and without a device exchange. If the implant is found to be positioned correctly, the grasper mechanism can be actuated to release the implant from the catheter.

### CROSS-REFERENCES TO RELATED APPLICATIONS

This application claims the benefit of provisional application No. 60/759,713 (Attorney Docket No.: 017534-003400US), filed on Jan. 17, 2006, the full disclosure of which is incorporated herein by reference.

### BACKGROUND OF THE INVENTION

The subject invention relates to the field of medicine and to the field of performing catheter-based therapeutic procedures within the body. In particular the invention relates to delivering implants or biologics into the airways of the lungs for interventional purposes.

Tubular structures exist in many organ systems within the body, such as in the circulatory system, the cardiovascular system, the reproductive system, gastrointestinal system, otolaryngology system, and the respiratory system. Specific structures may include airways, fallopian tubes, bile ducts, blood vessels, or the esophagus. Further, artificially created lumens can exist. Tubular systems can be accessed from outside the body for the therapeutic or diagnostic purposes through channels that naturally communicate with the outside, or through artificially created channels.

In the case of delivering implants such as stents, plugs, coils, baskets, filters, valves, grafts, prosthesis', drugs, drug reservoirs, biologics, or pumps. Typically, the access catheter is placed percutaneously or transluminally from outside the patient to inside a lumen or cavity of the target organ. In the case of a cardiovascular implant, the access catheter may be an introducer that percutaneously penetrates the femoral artery or vein. An interventional catheter is then placed through the introducer and fed to the target heart location in order to perform the intervention, such as stent delivery. In the case of the bronchial tree in the lung, a bronchoscope is used as the access catheter or introducer through which the interventional instruments are passed, such as stent delivery catheters or biopsy forceps.

A significant drawback of the systems and techniques currently available is the ability to maintain a hold of the stent prior to fully releasing it which can result in an inaccurately placed stent that can not be removed or re-positioned or requires a device exchange to do so. A device exchange can lead to extended procedure time as well as frustration to the operator. In some circumstances, the anatomy can make it difficult or impossible to retrieve the inaccurately placed stent within the acceptable procedural time limits even with the device exchange.

Of particular interest to the present invention, self-expanding flow restrictive elements may be delivered to airways of the lung for performing endobronchial lung volume reduction surgery. By occluding the airway to a diseased lung segment, that segment is deprived of air which in turn reduces the hyperinflation of the lungs and can restore some lung function. Typically, the flow restrictive element is a self-expanding flow restrictive stent comprising an elastic scaffold covered by a gas impermeable barrier. In other cases, the flow restrictive element comprises a one-way valve which allows air to be expelled from the isolated lung segment but prevents air from entering said segment.

Heretofore, such flow restrictive stents and other flow restrictive elements have been delivered by positioning a delivery catheter or sheath, expelling the flow restrictive element from the sheath, and allowing the flow restrictive element to become anchored or positioned at a desired location immediately upstream of the diseased lung segment. While generally successful, removing, repositioning, and other manipulation of the flow restrictive element after the initial delivery step have been problematic.

For these reasons, it would be desirable to provide improved methods and systems for releasing, positioning, and optionally repositioning flow restrictive stents and other elements within an airway of the lungs. In particular, it would be desirable to provide methods and systems which permit the flow restrictive element to be initially released from a delivery sheath or other tool, be temporarily positioned at a location in the airway, and thereafter be repositioned or retrieved if the initial positioning is unsatisfactory. At least some of these objectives will be met by the inventions described hereinbelow.

### SUMMARY OF THE INVENTION

According to the present invention, methods and systems are provided for positioning flow restrictive elements in an airway of the lungs. In particular, the methods and systems are suitable for performing endobronchial lung volume reduction surgery (LVRS) by positioning the flow restrictive element in airways leading to a diseased lung segment. A diseased lung segment could be a lobe segment or a sub-lobular segment, where the flow restrictive element is located in the airway leading to the diseased segment to block air from entering that segment. The element may be a fully occluding element, such as a self-expanding scaffold covered with a gas permeable barrier, such as described in detail in U.S. Pat. No. 6,997,981, or may comprise a one-way valve, as described in U.S. Pat. No. 6,527,761, the full disclosures of which are incorporated herein by reference. In presently preferred embodiments, the flow restrictive element may comprise discrete openings, passages, tubes, orifices, or other features which allow for bidirectional flow at a relatively low rate between the feeding airway and the target lung segment. Such flow controlling elements are described in copending application No. 60/780,577 (Attorney Docket No. 017534-003500US), the full disclosure of which is incorporated herein by reference.

Methods according to the present invention for positioning a flow restrictive element in an airway comprise advancing the flow restrictive element from a sheath. The flow restrictive element is adapted to self-expand as it is released from the sheath. After the element has been advanced from the sheath, it may either be released into the airway where it may remain as a permanent implant or may be later retrieved, or it may be directly drawn back into the sheath to permit removal or repositioning.

In the exemplary embodiments, advancing comprises moving a grasping tool which releasably holds the flow restrictive element through a lumen of the sheath. Releasing typically comprises disengaging an attachment member on the grasping tool from the flow restrictive element. Optionally, the method may further comprise re-engaging the attachment member with the flow restrictive member. After such re-engagement, the grasping tool may be used to either reposition the flow restrictive element or to draw the flow restrictive element back into the sheath to permit further repositioning or removal.

In a second aspect of the present invention, a system comprises a self-expanding flow restrictive element adapted for release to occlude an airway in a lung. Such element may either be a fully flow restrictive element, such as a self-expanding scaffold covered by an air impermeable barrier, or may alternatively be a one-way valve structure which when implanted permits air to be expelled from the isolated lung region but which prevents air from re-entering said lung region. The system further comprises a delivery sheath having at least one lumen adapted to recede and constrain the self-expanding flow restrictive element. The grasping tool is provided and includes an attachment member which releasably holds the flow restrictive member. Thus, the grasping tool may be axially advanced through the delivery sheath lumen to position the flow restrictive element in the airway. The attachment member may then be actuated to release the flow restrictive member at the location where it has been positioned. Alternatively, the grasping tool may be drawn back into the sheath in order to re-constrain and remove the self-expanding flow restrictive element from the airway.

### BRIEF DESCRIPTION OF THE DRAWINGS

FIGS. 1, 1A, and 1B describe typical biopsy forceps modified with a feature to hold the stent bushing.

FIG. 2 describes a modified biopsy forceps holding a stent inside the sheath of the delivery system.

FIG. 3 describes a stent unsheathed but not yet released from the grapser mechanism of the delivery system.

FIG. 4 describes a stent unsheathed and released from the grasper mechanism of the delivery system.

FIG. 4A describes an alternative configuration of the first embodiment in which the biopsy forceps or grasper jaws are biased in the open position.

FIGS. 4B -4D describe the unsheathing steps of the alternative configuration of the first embodiment in which the biopsy forceps jaws are biased in the open position.

FIGS. 5 and 5A-5E describe alternative multi-prong grasper embodiments of this invention.

FIG. 6 describes the multi-prong grasper embodiment of this invention holding a stent inside the sheath of the delivery system.

FIG. 7 describes the multi-prong grasper embodiment of this invention unsheathing the stent but still maintaining a hold of the stent.

FIG. 8 describes the muti-prong grasper embodiment of this invention with the stent unsheathed and released.

FIG. 9 describes a loop snare embodiment of this invention.

FIG. 10 describes details of the distal end of the loop snare.

FIGS. 11A and 11B describe the loop snare embodiment of this invention maintaining a hold of the stent that has been unsheathed.

FIG. 12 describes the loop snare embodiment of this invention with the stent unsheathed and released.

FIGS. 13, 13A and 13B describe a keyed lock embodiment of this invention showing the detailed key features of the stent and the shaft end.

FIGS. 14A and 14B describe the keyed lock embodiment of this invention showing the collapsed stent sheathed and the keyed lock engaged.

FIG. 15 describes the keyed lock embodiment of this invention showing the stent unsheathed but still attached to the delivery system.

FIGS. 16A-16C describe the keyed lock embodiment of this invention showing the rotational action required to disengage the delivery system from the stent.

FIGS. 17A-17C describe details of possible keying configurations.

FIG. 18 describes the internal latch embodiment of this invention.

FIGS. 19A and 19B describe the details of the distal end of the internal latch embodiment of this invention.

FIGS. 20A-20C describe the details of the distal end of the outer shaft latches of the internal latch embodiment of this invention.

FIGS. 21A-21C describe the engagement of the stent with the internal latch embodiment of this invention.

FIG. 22 describes the sheathed stent inside the internal latch embodiment of this invention.

FIGS. 23A-23C describe the internal latch embodiment of this invention disengaging the stent from the delivery system.

FIG. 24 describes the hook and slot embodiment of this invention.

FIGS. 25A-25C describe the details of engaging the stent with the hook and slot embodiment of this invention.

FIGS. 26A-26C describe the unsheathing step with the hook and slot embodiment of this invention.

FIGS. 27A-27C describe the step required to release the implant from the hook and slot embodiment of this invention.

FIG. 28 describes the controlled stent expansion embodiment of this invention.

FIGS. 29A-29C describe the sequence of expansion steps in the controlled expansion embodiment of this invention.

FIGS. 30A and 30B describe the controlled stent expansion embodiment of this invention with the unsheathed stent still in its collapsed state and not yet released.

FIGS. 31A-31C describe the controlled stent expansion embodiment ofthis invention with the unsheathed stent expanding and being released from the delivery system.

FIGS. 32A-32C describe a steerable tip hook embodiment of this invention.

FIG. 33 describes the distal end of the steerable tip hook embodiment of this invention with a sheathed stent.

FIG. 34 describes the steerable tip hook embodiment of this invention with the stent unsheathed but not yet released.

FIGS. 35A and 35B describes the steerable tip hook embodiment of this invention releasing the unsheathed and expanded stent.

FIG. 36 describes the screw attachment embodiment of this invention.

FIG. 37 describes the distal end of the screw attachment embodiment of this invention with the stent sheathed and still attached to the delivery system.

FIG. 38 describes the screw attachment embodiment of this invention with the stent unsheathed and still attached to the delivery system.

FIG. 39 describes the screw attachment embodiment of this invention with the stent unsheathed and released from the delivery system.

FIG. 40 describes the spring snare embodiment of this invention.

FIGS. 41A and 41B describe the details of the distal end of the spring snare embodiment of this invention.

FIG. 42 describes the details of the distal end of the spring snare embodiment of this invention with the stent sheathed and attached to the delivery system.

FIGS. 43A and 43B describe the spring snare embodiment of this invention releasing the unsheathed stent from the delivery system.

### DETAILED DESCRIPTION OF THE INVENTION

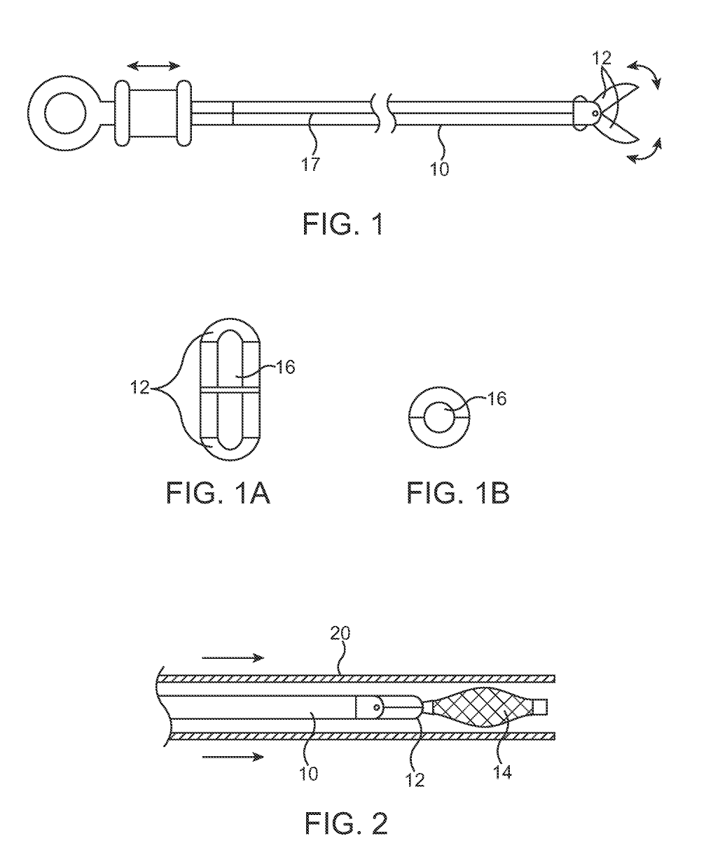

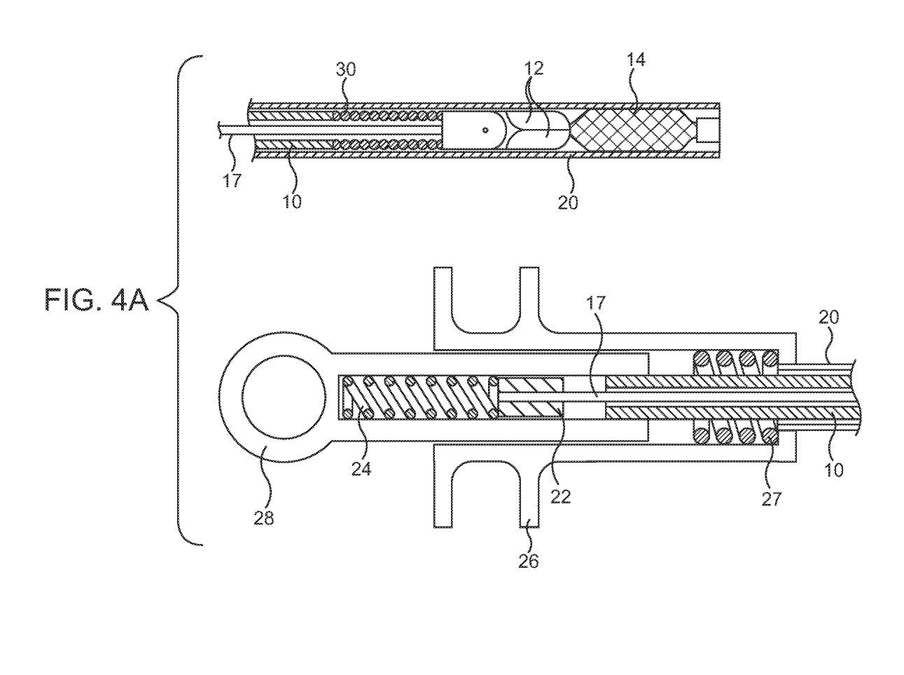

FIGS. 1, 1A and 1B describe typical biopsy forceps 10 used in today's bronchoscopy diagnostics and interventions with a modification to jaws 12 to allow them to hold a flow restrictive stent 14 (FIG. 2) without increasing its closed jaw profile. Typical biopsy forceps have hollow jaws and a round, hemispherical distal tip that is closed when the jaws are closed. FIG. 1 illustrates a modification made to the forceps. FIG. 1 does not show the outer sheath that houses the collapsed stent. A hole or opening 16 is formed in the jaws at the distal tip, as seen in FIGS. 1A and 1B. This hole or cut out provides for clearance with a feature on the stent but interferes with a more proximal feature on the stent. This interference enables the forceps to maintain a hold of one end of the stent. The opening 16 of the jaws 12 and this cut out allow for a feature on the stent, typically a bushing 13 (FIG. 4) to be held inside the jaws without interfering with the jaws closing. This allows the closed jaw profile to not increase while holding the implant. It is important to maintain a low profile so that the overall device maintains a low profile enabling it to fit into and maneuver inside the small tubular structures of the body.

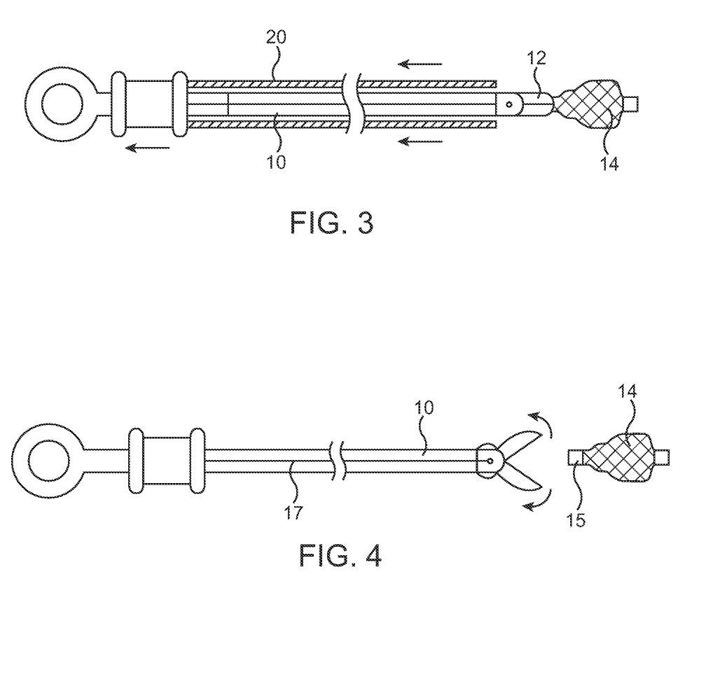

FIG. 2 illustrates a distal end of the forceps 10 of this invention, a stent delivery system utilizing the modified biopsy forceps described in FIG. 1. In FIG. 1, the implant is a stent in its collapsed state housed inside a sheath. The biopsy forceps with the before described modification can maintain a hold of the stent inside a sheath 20 without increasing its overall closed jaw profile. The biopsy forceps in this configuration maintains a hold of the stent 14 as the stent is axially advanced to assume an increased diameter, as shown in FIG. 3. The stent 14 has been completely unsheathed but the biopsy forceps 10 still maintain a hold of the stent. At this stage, the operator has several options. The options include completely removing the stent, repositioning or dragging the stent 14, re-sheathing the stent then repositioning or removing it, or completely release the stent. Which option the operator chooses will be based on the assessment of the placement of the stent. If the placement is assessed to be acceptable, the option for completely releasing the stent is chosen. If the placement is assessed to be unacceptable, then one of the other options is chosen at the discretion of the operator.

The final release stage of the stent delivery is shown in FIG. 4. The outer sheath 20 that housed the collapsed stent is not shown in FIG. 4. At this point, the forceps 10 or catheter was positioned, the stent 14 was unsheathed, and the final position of the stent was found to be acceptable so the jaws were opened to facilitate final release of the stent. The jaw opening mechanism could be a conventional wire linkage system that allows for jaw opening and closing with a push or pull of a central wire.

FIG. 4A describes an alternative configuration of the release mechanism. The modified graspers or biopsy forceps 10 used in the first embodiment can also have an additional mechanism in the proximal end that biases the jaws to be in the open position. A pull wire 17 of the graspers is designed to be “pull to close”. A piston 22 is fixed to wire 17 and combined with a compression spring 24 or a “jaw opening spring” maintains a “push” force on the wire. This type of loading forces the jaws to open when unconstrained. The loading can be low enough that the outer sheath 20 constrains the jaws 12 to the closed position but high enough to allow the jaws 12 to open freely when unsheathed. This greatly simplifies the hand manipulations required of the operator because the steps to unsheathe the stent will also unsheathe the jaws. It allows for a more intuitive and automated release of the stent. FIG. 4A also shows the actuation barrel 26 of the proximal handle housing a final release spring 27. This spring comes into play at the end of the unsheathing stroke. An actuation ring 28 is actuated with the actuation barrel 26 using a syringe action like maneuver. The actuation ring 28 will come into contact with the final release spring 27 and begin loading it at the same time the unsheathing step is complete. The final release spring maintains the jaw position with a compressive load to be completely sheathed and therefore the jaws are closed. This spring must be defeated with a continuation of the same syringe like stroke to allow the jaws to be unsheathed and open releasing the stent. A compression coil 30 which allows for the appropriate column strength for the grasper for unsheathing but also provide the necessary flexibility for tortuous anatomy.

FIGS. 4B-4D provides a detailed view of the alternative configuration of the first embodiment. The three illustrations show the steps for release. Fig, 4B shows the stent 14 completely unsheathed but still attached to the grasper jaw 12. The final release spring housed in the proximal handle is maintaining a load on the graspers to keep it sheathed. With the jaws sheathed, the jaws are closed maintaining a hold of the stent. The next illustration shows the grasper tip unsheathed, jaws open, and releasing the stent. The final release spring was defeated allowing the jaws to be unconstrained by the outer sheath, releasing the stent. The unsheathing of the jaws 12 combined with the biasing load of the jaw opening spring, allows the jaws to open without any additional hand manipulations from the operator. The last illustration shows the effect of the operator releasing the syringe action on the proximal handle. The final release spring takes over and draws the graspers back into the sheath. The re-sheathing of the jaws collapses them back into the closed position and returns them into the outer sheath.

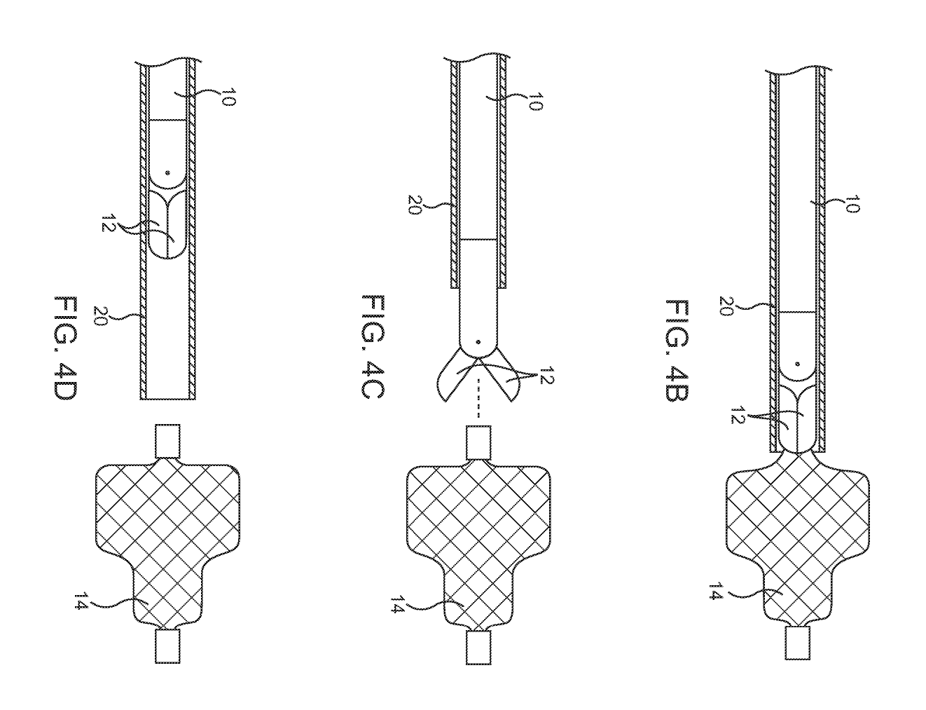

FIGS. 5 and 5A-5D illustrate alternative grasper configurations according to the present invention. The graspers are composed of multiple prongs 36 that are spring loaded to be biased open in the unconstrained state. When pulled from an unconstrained configuration (FIG. 5A) into a funnel shaped, collet type component 40 (FIG. 5B), the prongs close down on an object radially. A wire 17 is attached to the prongs which allow for a pulling the prongs into the funnel 40, or pushing the prongs out of the funnel. Pulling the prongs into the funnel allows for closing and pushing the prongs would allow for opening. This embodiment may include but is not limited to two prongs (FIG. 5C), three prongs (FIG. 5D), or four prongs (FIG. 5E).

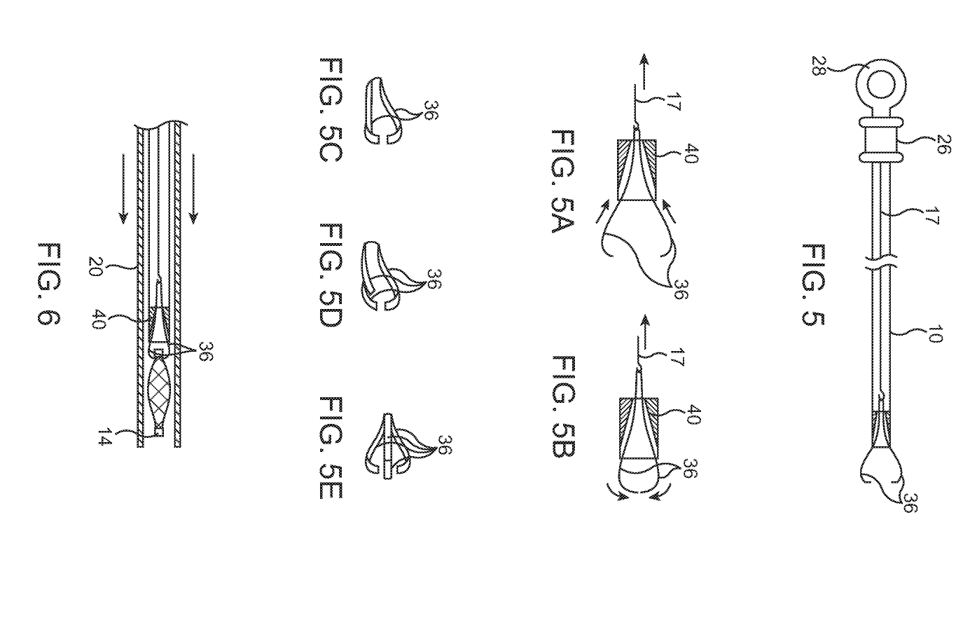

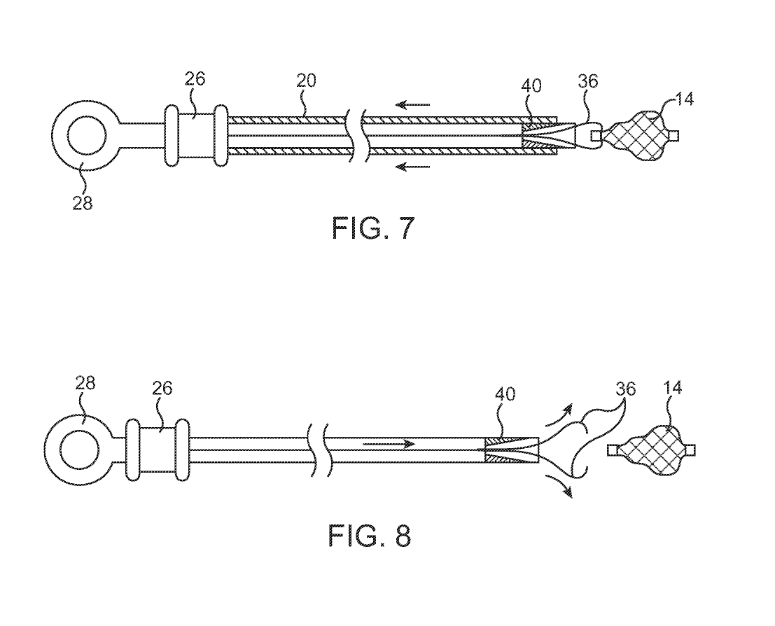

FIG. 6 describes a cross-sectional view of the distal end of the prong embodiment of this invention. The stent is in its collapsed state housed inside the sheath of the delivery system. The prongs of the grasper are pulled into the funnel allowing it to maintain a hold of the stent. The grasper would also be required to resist the unsheathing forces of the next steps with column strength. The stent 14 is fully unsheathed and is in its expanded state but not yet released. The multiple prong grasper is in its closed state and is maintaining a hold of the stent. At this point, the operator has several options as previously stated. The stent 14 is fully unsheathed and in its expanded state in FIG. 7 and fully released from the delivery system in FIG. 8.

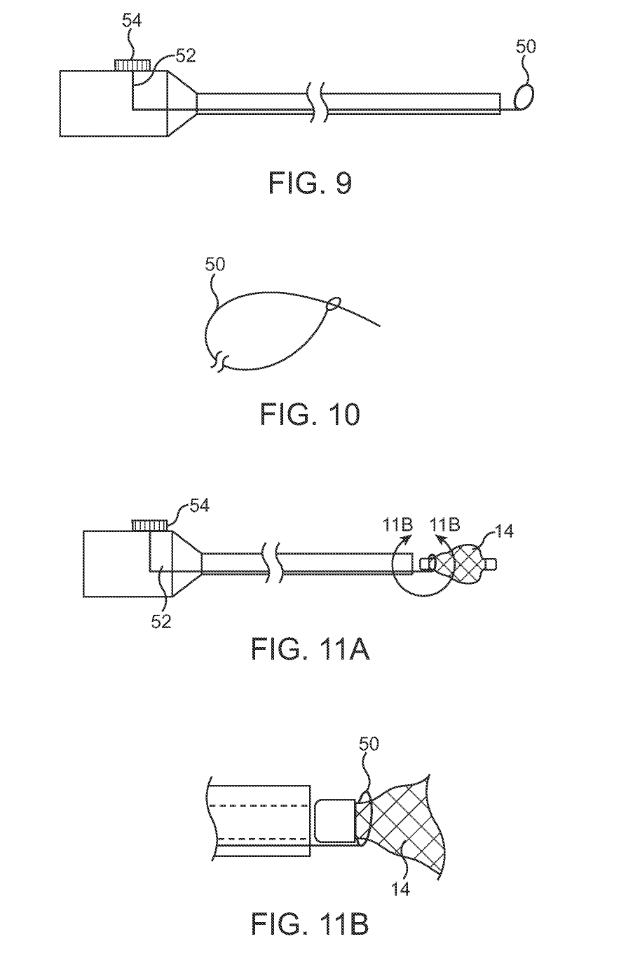

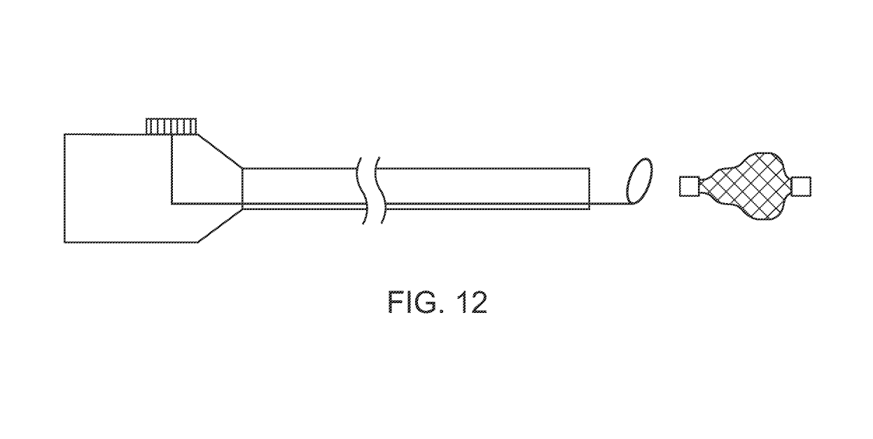

FIG. 9 describes the third embodiment of this invention. A loop snare 50 is formed from a wire or string and is configured to snare the stent 14. A proximal end 52 of the wire or string is attached to an actuator handle 54 that enables remote tightening or loosening of the snare on the stent. FIG. 10 illustrates the loop 50 of the distal end of the wire or string in detail. The loop is configured in such a way that allows for tightening of the snare when placed in tension and loosening of the snare when tension is released and/or compression is placed on it. One means of accomplishing this is to place an eyelet 51 at the end of the wire or string and then to run the other end of the wire or string through the eyelet. Pulling on the wire or string tightens the snare and releasing or pushing the wire or string loosens the snare. The loop 50 snaring a proximal end of the stent 14 is shown in FIGS. 11A and 11B. The loop tightens down on the proximal end of the stent snaring it. This action maintains a hold of the stent to the delivery system. Tension on the wire or string tightens the loop and maintains a hold of the stent. Releasing the tension or applying compression on the wire or string loosens the loop which allows the stent to be released from the delivery system. The stent in FIGS. 11A and 11B is completely unsheathed but the snare is maintaining a hold of it. As shown in FIG. 12, the stent 14 has been fully unsheathed and the position has been assessed to be adequate. The last step is to release it from the delivery system. Tension on the wire or string is released or compression is applied which loosens the loop. The loop diameter increases and the snare is removed from the stent. The stent is now released from the delivery system.

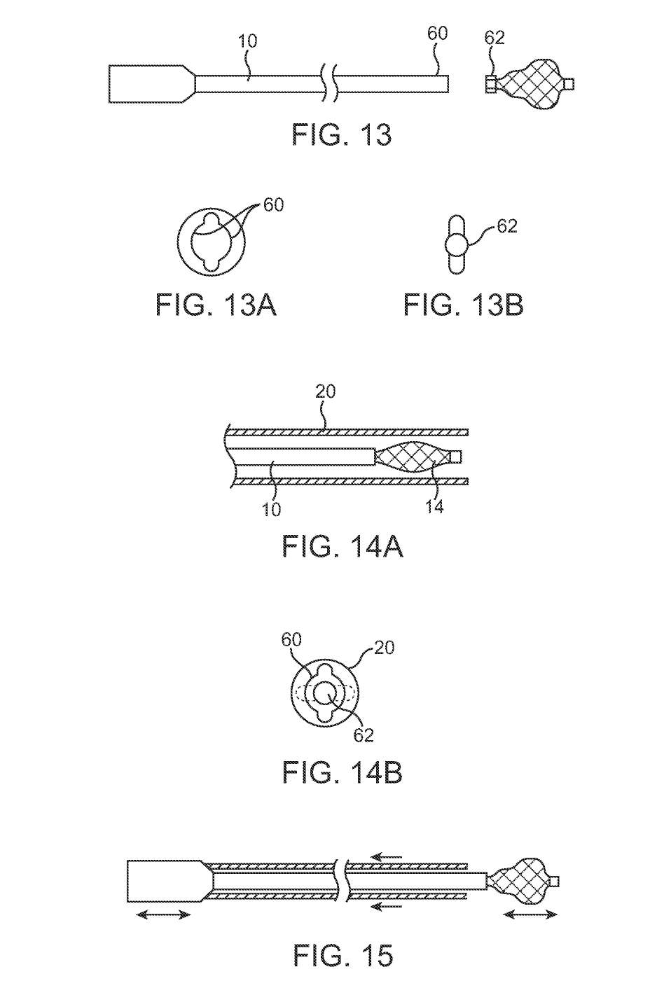

As shown in FIGS. 13, 13A and 13B, the distal end 60 of the catheter 10 and the proximal end 62 of the stent 14 have mating or keyed features that when engaged, are locked together. To engage the stent to the delivery system, the proximal end of the stent would be inserted into the mating feature of the delivery system then rotated 90 degrees to lock it axially. An undercut type of feature prevents the stent from detaching from the delivery system until it is rotated 90 degrees from the locked position.

As shown in FIGS. 14A and 14B, the stent 14 is in its collapsed state and fully sheathed. The center push rod is the component of the delivery system that has the mating engagement feature. The stent 14 is engaged into the center push rod and this engagement is what allows the delivery system to maintain a hold of the stent 14. The center push rod not only serves the function of engaging the stent but also serves the function providing column strength to resist the unsheathing forces during that portion of the delivery.

As shown in FIG. 15, the delivery system is positioned in the target location and the sheath 20 retracted relative to the stent 14. The column strength of the center push rod resisted the frictional unsheathing forces and this allows the stent to maintain position during the unsheathing process. While the stent is fully unsheathed, it is still connected to the delivery system with the keyed lock mechanism. At this step, the unsheathed position of the stent is assessed to determine if the stent should be released from the delivery system or should be removed from the body to be reattempted.

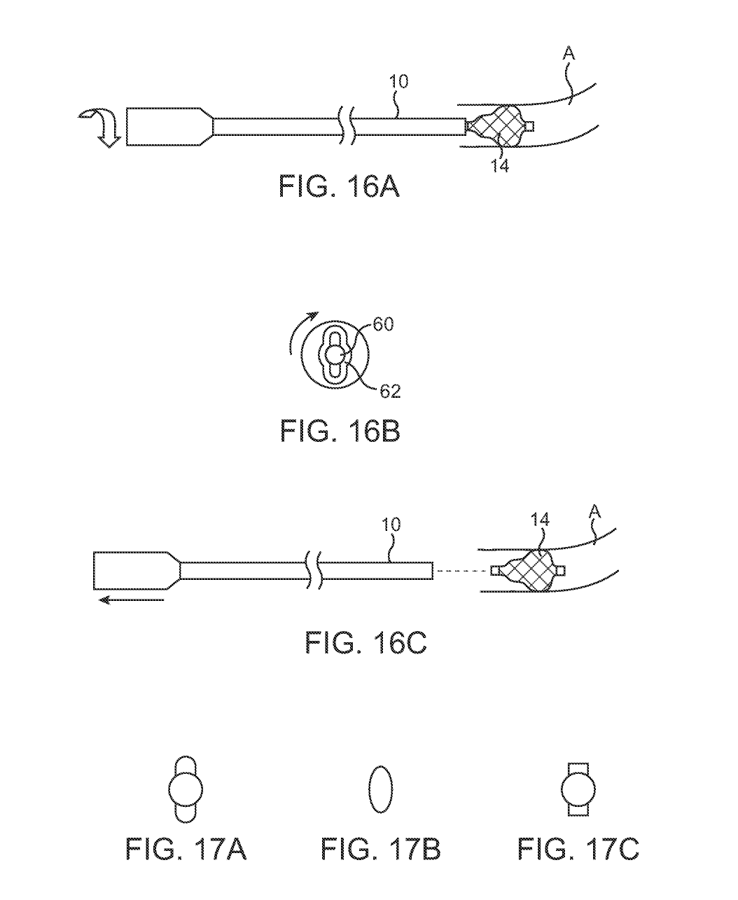

As shown in FIGS. 16A-16C, after assessment of the stent is complete and it is determined that the final position is adequate, the stent 14 is released into airway A. A 90 degree rotation of the shaft is actuated remotely at the proximal handle. This rotation is transmitted through the delivery system and the keyed lock mechanism reaches a disengagement point. This disengagement point is where the stent lock feature mates with the center push rod feature and there exists no interference. With no interference, the stent can separate from the center push rod. At this point, the delivery system is detached from the stent.

FIGS. 17A-17C describes three different keying configurations that can be used for the fourth embodiment of this invention. One configuration has a center round hole with rounded slots at the 90 degree position and the 270 degree position (FIG. 17A). The next configuration has a more elliptical or oval shape (FIG. 17B). The last configuration shown has a center round hole with square slots at the 90 degree position and the 270 degree position (FIG. 17C). Each of these configurations operates similarly in that a hole of these shapes will accept a smaller rod of these shapes. A 90 degree rotation of the rod with respect to the hole will engage the two components and provide an interference. This interference allows the two components to stay attached. Just as a 90 degree rotation allows the two components to engage, another 90 degree rotation allows the two components to disengage. Disengagement can occur when the longer features located at 90 degrees and 270 degrees are aligned with respect to the two components. At this position, there is no interference and the two components can be separated.

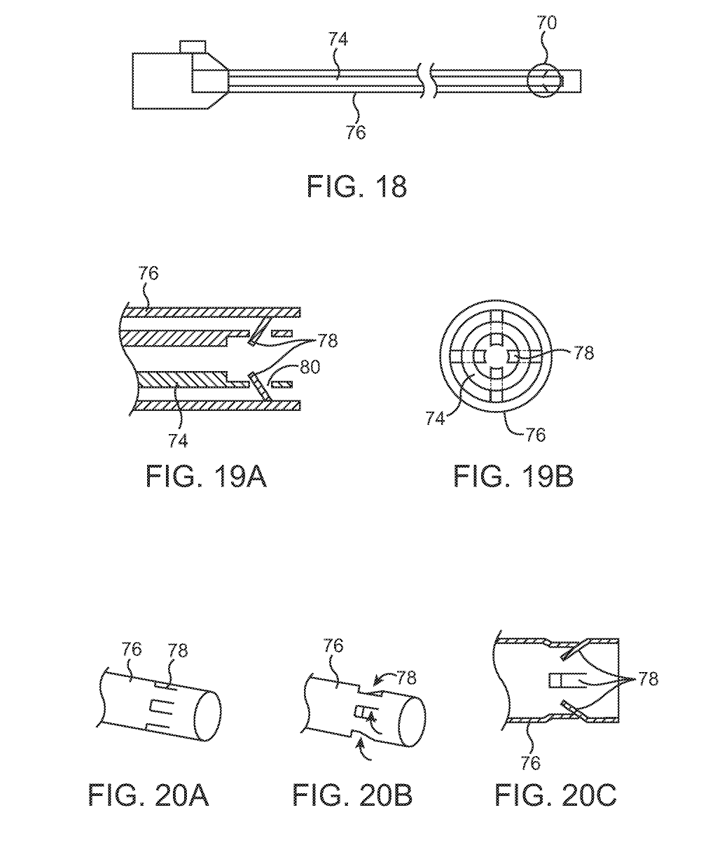

FIG. 18 describes an internal latch system 70. This system has an outer sheath (not shown) that houses the collapsed stent and a central push rod 72 that on the distal end has a mechanism to attach to the stent. The center push rod has two components, an inner shaft 74 and the outer shaft 76. The center push rod must provide column strength to resist the unsheathing forces and also provide a means of attaching to the stent. In this embodiment, the mechanism for attachment to the stent is comprised of retractable latches that provide interference with a mating feature on the stent.

FIGS. 19A and 19B illustrate the distal end of the center push rod 72 of the internal latch system. The outer shaft 76 shown is that of the center push rod. This outer shaft 76 contains retractable latches 78. These latches provide an interference to hold the stent to the delivery system. The inner shaft 74 provides windows 80 which receive the latches. The inner shaft is positioned such that the latches are in the engaged position. To retract the latches for disengagement from the stent, the inner shaft is retracted. The retraction of the inner shaft allows the windows to contact the latches and force them outward, retracting the latches from the engaged interference position (FIG. 19B).

FIGS. 20A-20C illustrate the distal end of the center push rod outer shaft of the internal latch embodiment of this invention. The outer shaft 76 of the center push rod has the latches 78 that engage and maintain a hold of the stent. These latches provide the necessary interference to hold the stent. FIG. 20 shows possible steps for fabrication of the latches. The first illustration shows the distal end of the outer shaft with cuts that begin the shape of the latches. This cut can be done with but not limited to laser cutting, wire EDM, plunge EDM, water jet, conventional machining, chemical etching, etc. The next illustration shows the cut latches being bent or formed inward to create the necessary interference. The cross-section shows the latches in its final state, cut and formed inward.

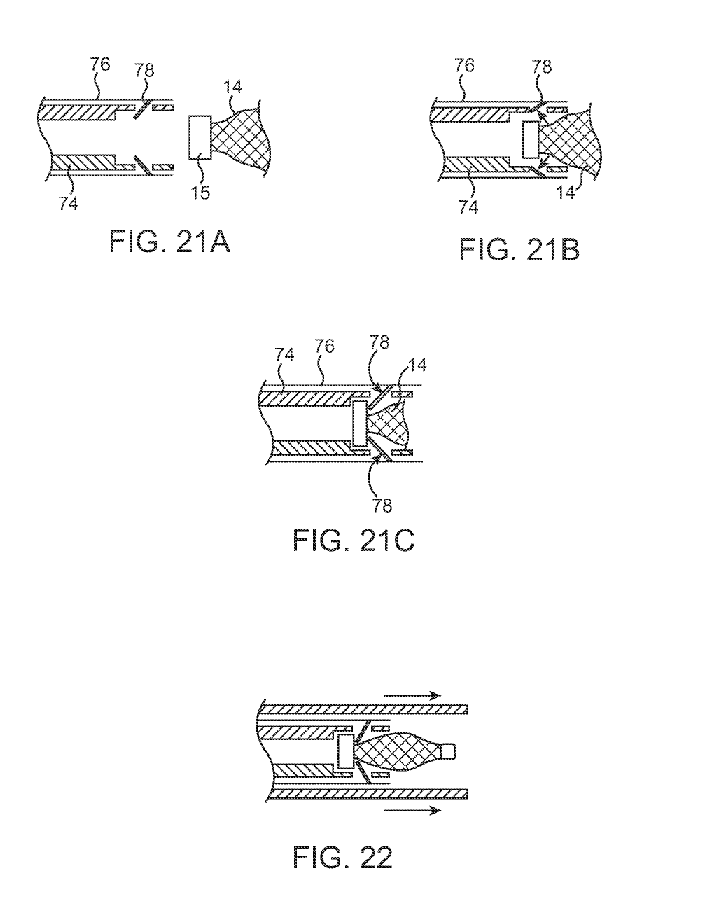

FIGS. 21A-21C illustrate operation of the internal latch embodiment of this invention. The outer shaft 76 and the inner shaft 74 of the center push rod are positioned in the engagement state. The stent 14 is then inserted into the outer shaft 76 which forces the latches 78 to flex outward for a brief moment. The latches are then in a sprung state until a relief feature in the stent is reached. When this relief feature is reached, the sprung latches snap into an engaged position which allows for interference with the mating feature of the stent. The stent 14 is now attached to the center push rod of the delivery system.

As shown in FIG. 22, the stent 14 is in the collapsed state and housed by the outer sheath 20 of the delivery system. The center push rod is in the engaged state and is attached to the stent 14. The latches 78 are protruding through the windows of the inner shaft 74, which are providing the necessary interference for attachment of the stent to the center push rod. From this stage, the stent is positioned in the target area with unsheathing to follow.

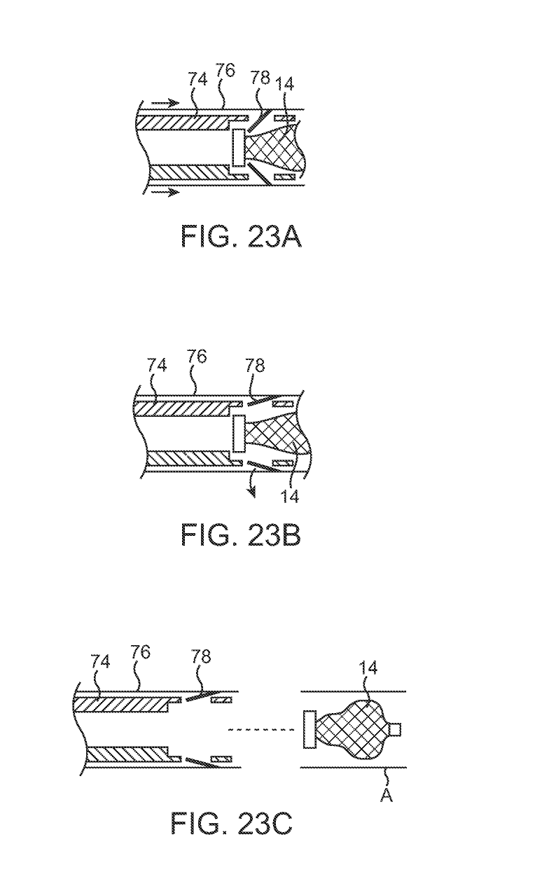

As shown in FIGS. 23A-23C, the latches 78 of the outer shaft 76 are in the engaged position and are maintaining a hold of the stent 14. To release the stent 14, the inner shaft 74 is retracted which in turn retracts the latches 78. The latches 78 are then disengaged from the stent 14 which allow the stent to be released from the delivery system into airway A.

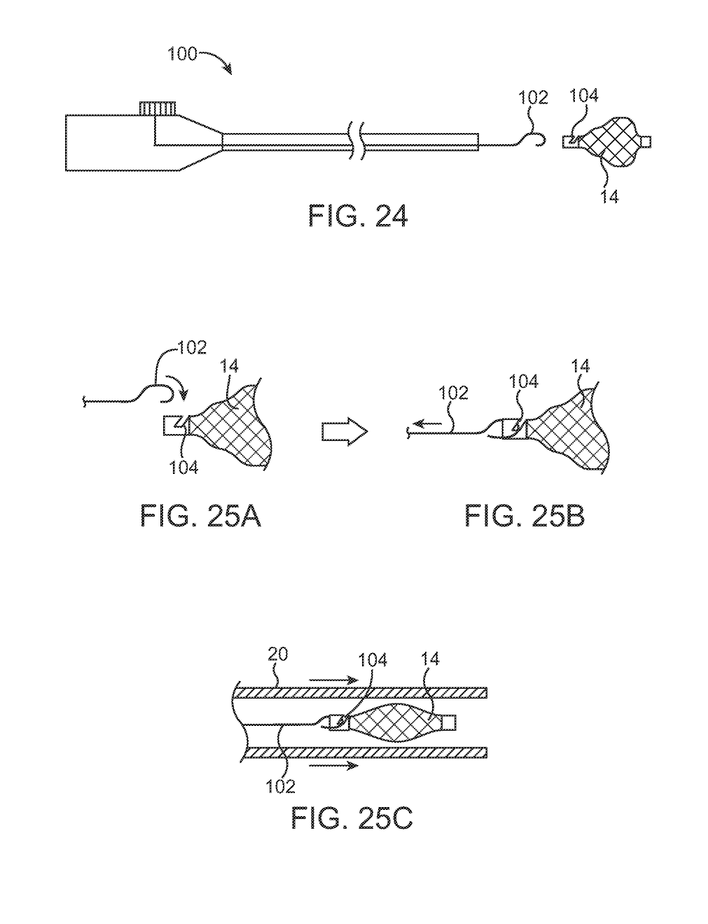

FIG. 24 illustrates a hook and slot delivery system. The delivery system 100 is shown to have released the stent 14 with a hook 102 disengaged from the stent. The stent 14 has a slot 104 in the proximal end that engages the hook. The hook 102, when engaged to the slot 104 of the stent, maintains a hold of the stent to the delivery system. The hook could alternatively be a closed loop or partially closed loop of various shapes. From this point on, when referring to this embodiment, a hook can be interchangeable with a loop. Tension placed on the hook 102 maintains engagement into the slot 104. Compression or distal motion of the hook 102 relative to the stent 14 will disengage the hook from the slot 104. The opposite configuration of can also be possible with the hook or loop feature on the stent and the slot feature on the delivery catheter.

FIGS. 25A-25C illustrate use of the hook and slot embodiment of this invention. The slot width allows for a close clearance fit of the hook. The angle of the slot is configured in such a way that when tension is placed on the wire, further engagement is achieved. Also, FIG. 25 shows the collapsed stent housed by the outer sheath and engaged to the hook.

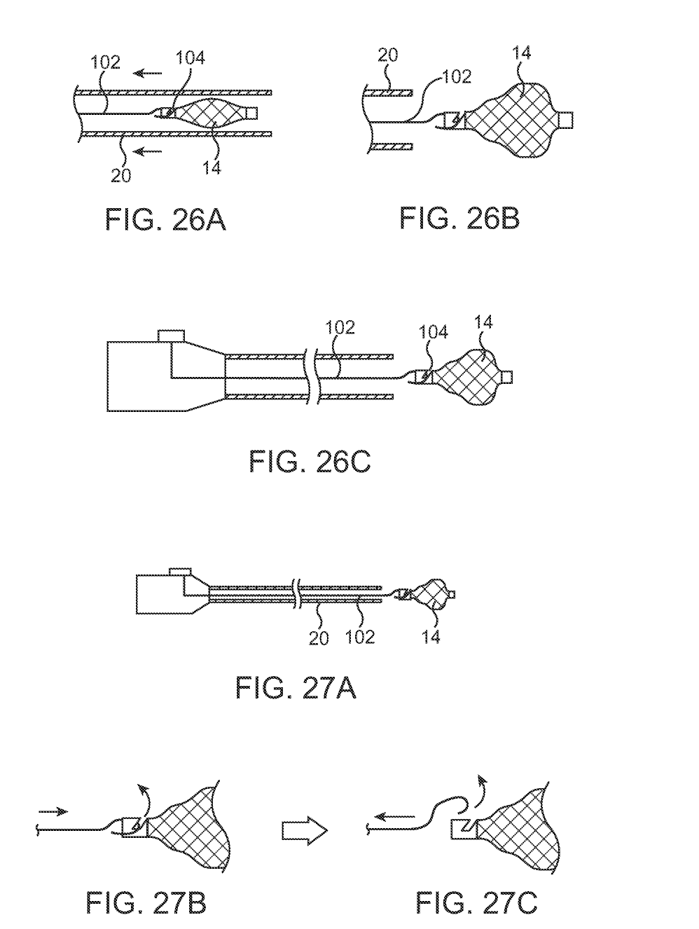

FIGS. 26A-26C illustrate the stent 14 in the collapsed state housed by the outer sheath 20, the engaged hook 102 component or push rod must provide column strength to resist the frictional unsheathing forces. When the delivery system is positioned in the target area, unsheathing the stent is the next step. During the unsheathing operation, the outer sheath 20 is retracted and the hook component 102 provides opposing forces which allows the stent 14 to maintain its position. With the hook still engaged, the delivery system maintains a hold of the unsheathed stent.

FIGS. 27A-27C illustrate the stent release step. If the placement of the unsheathed stent is found to be adequate, the hook 102 may be disengaged from the stent slot 104. This is achieved with a motion of the hook 102 relative to the stent along the ramp of the slot 104. A remote action at the proximal handle sends the hook wire distally forcing the hook 102 to travel up the ramp of the slot. This motion allows the hook to disengage from the slot. A rotation of the hook 102 may also perform this disengagement function or a proximal axial motion combined with a rotation. This disengagement of the hook 102 from the stent 14 releases the stent from the delivery system.

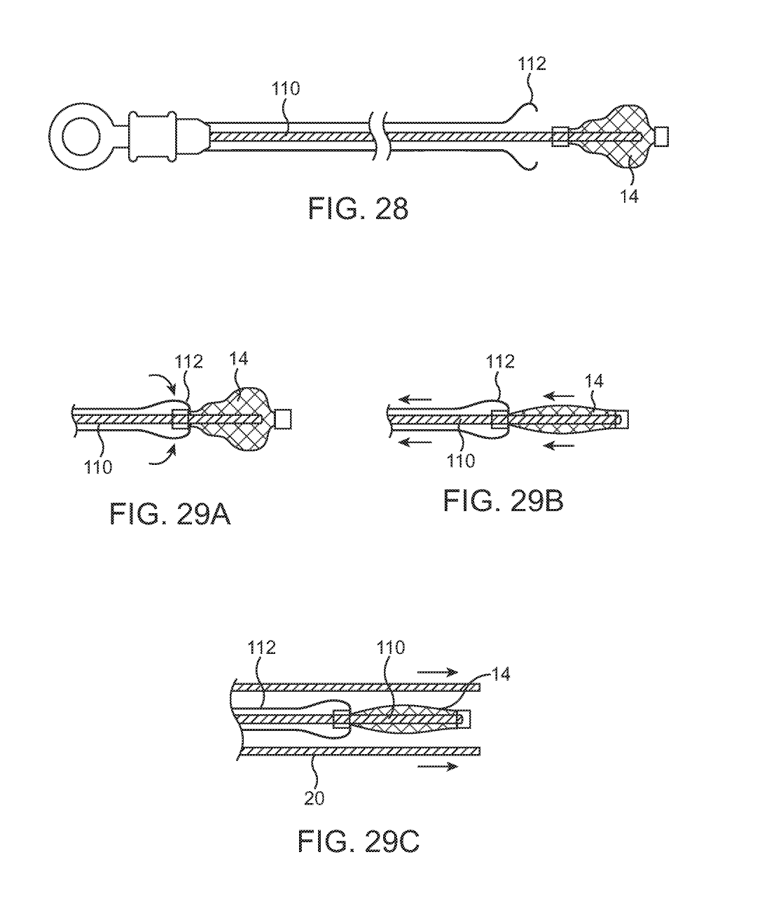

FIG. 28 illustrates an unsheathed stent 14 being released from a grasper-type mechanism 112 to the delivery system. This embodiment enables the operator to not only have control of when to release the stent 14 but also control of the expansion of the stent. A center rod 110 travels through the stent 14 and engages an internal feature at the distal end of the stent. On the proximal end of the stent, a grasper mechanism maintains a hold of a feature on the stent. With tension on the grasper mechanism and compression on the center rod, the self-expanding stent 14 is made to collapse. With release of tension on a grasper mechanism 112 and release of compression the center rod, the stent is free to expand. Controlling the release of tension and compression on these mechanisms allows for control of the expansion of the stent.

FIGS. 29A-29C illustrate the grasper mechanism holding the proximal end f the stent while a center push rod runs coaxially through the stent. The center push rod bottoms out at the distal end of the stent. While the grasper 112 holds on to the stent 14, compression is placed on the center push rod 110 with concurrent tension on the grasper to collapse the stent radially. In the collapsed state, the stent is housed by outer sheath 20 with the grasper maintaining a hold of it. The tension on the grasper and compression on the push rod may or may not be required to keep the stent in the collapsed state if the stent is completely housed by the outer sheath.

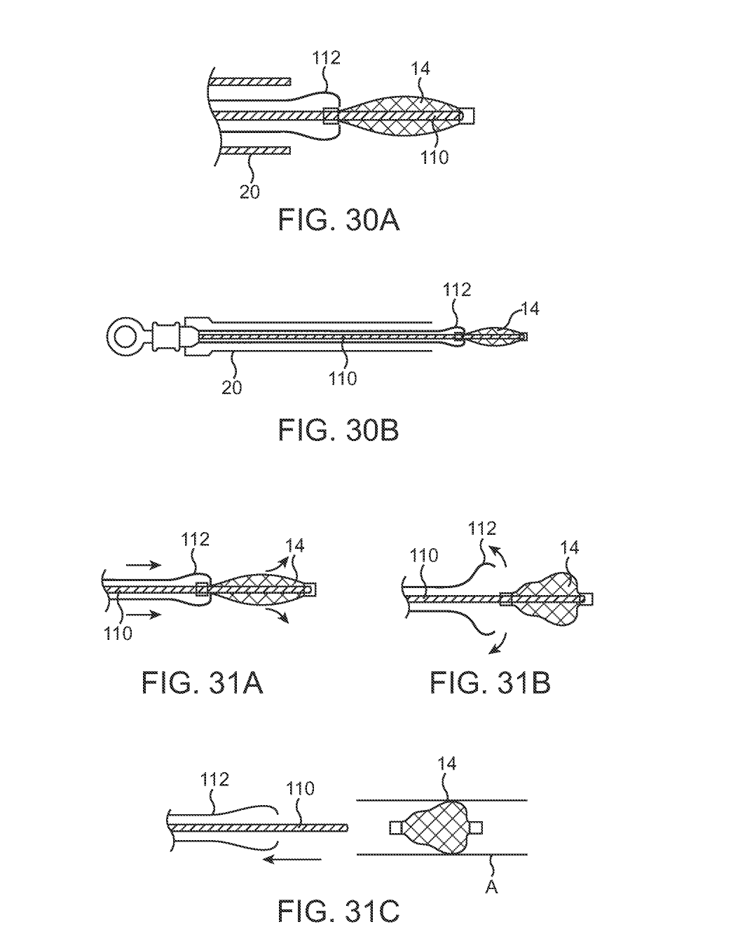

FIGS. 30A and 30B illustrate the unsheathing step of the controlled stent expansion embodiment of this invention. The delivery system is positioned in the target location and the sheath 20 is retracted. A mechanism is necessary to provide column strength to oppose the frictional unsheathing forces. In this embodiment it is either the center push rod, or the grasper or both. This embodiment enables the operator to unsheathe the stent and maintain the stent's collapsed state. This embodiment provides another level of control to the user, that is controlling the timing and the rate of expansion of the stent. The combination of the tension placed on the grasper and the compression placed on the center push rod, or the push/pull action, can be used to expand or collapse the stent. With the stent fully unsheathed, the push/pull action can be used to expand the collapsed stent, collapse the expanded stent, or partially expand the collapsed stent, or partially collapse the expanded stent. This level of control is desirable because it enables the operator to reposition the stent several times, without a device exchange, until a satisfactory placement is accomplished.

FIGS. 31A-31B illustrate controlled stent expansion. The stent 14 is shown to be fully unsheathed and expanding. The push/pull action is being used to expand the stent 14 in a highly controlled manner. Upon satisfactory placement and expansion, the operator makes the decision to release the unsheathed, expanded stent 14 from the delivery system into airway A. If the placement was found to be unsatisfactory, then prior to releasing the stent, the option of using the push/pull action to collapse the stent and reposition it was available.

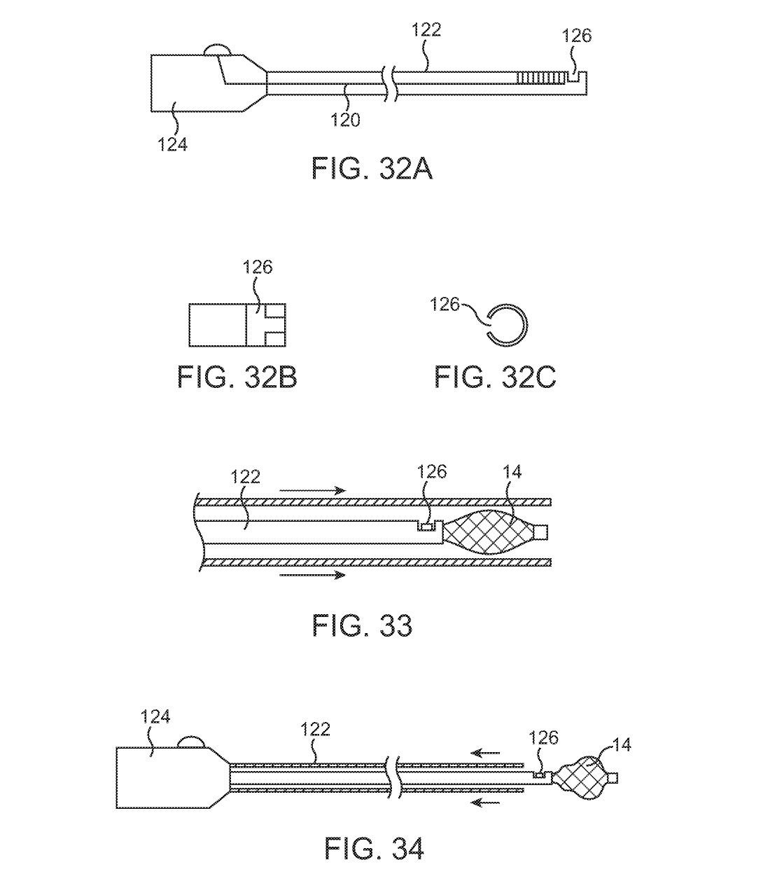

FIGS. 32A and 32B illustrate a steerable tip hook delivery system. A central tube or rod 120 with a control wire 122 is linked to the proximal handle 124. The distal end of the central tube has a notch 126 that engages the proximal end of the stent. The notch 126 allows the stent to slide in from the top direction but is trapped axially due to the interference that is created from the proximal end of the stent and the notched central tube. FIG. 32C is an end view showing the notch 126.

FIG. 33 illustrates the notched tip 126 of the central tube 122 or rod engaged with proximal end of the stent 14 creating an attachment between the stent and the delivery system. The central tube 122 or rod provides the stent attachment function but also provides the necessary column strength for opposing the unsheathing forces.

FIG. 34 illustrates the stent 14 positioned in a target location and the outer sheath retracted allowing the stent to expand. The central tube 122 or rod with the notch 126 is shown to be still engaged to the stent 14 allowing the operator the options of repositioning, re-sheathing then repositioning, re-sheathing then removing, or just completely the removing the stent as stated previously.

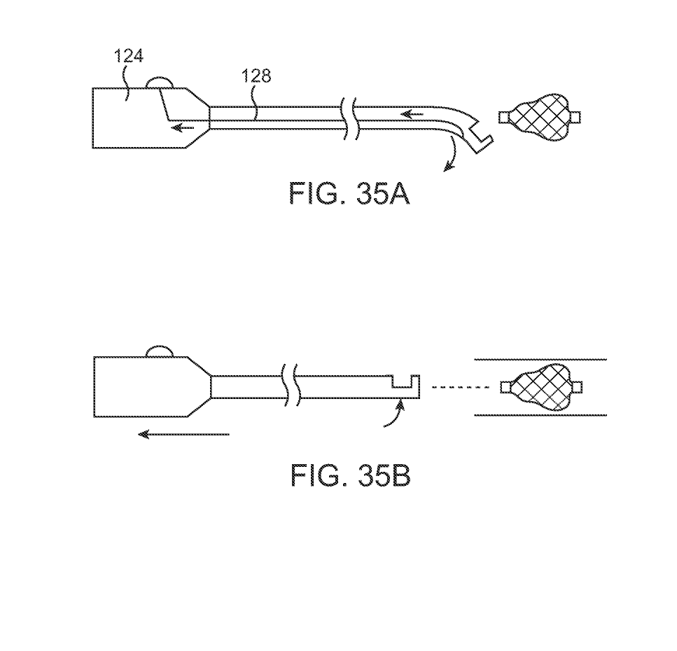

FIGS. 35A and 35B illustrate a control wire 128 actuated remotely from the proximal handle causing the tip of the central tube 122 or rod to articulate and steer away from the unsheathed and expanded stent. This articulation maneuver is how the delivery system detaches itself from the stent 14. After the delivery system detaches from the stent 14, the proximal handle 124 is actuated to return the central tube 122 distal tip to its original position.

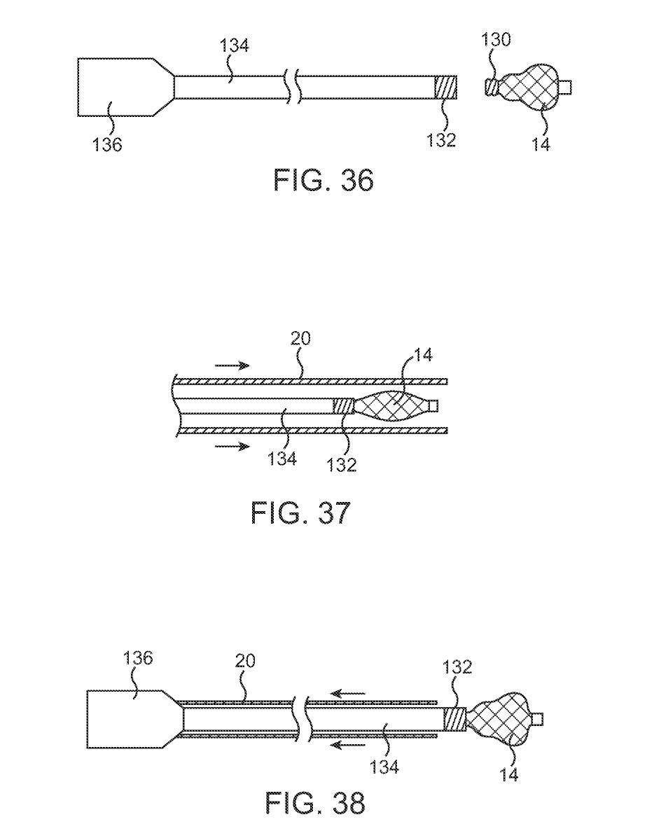

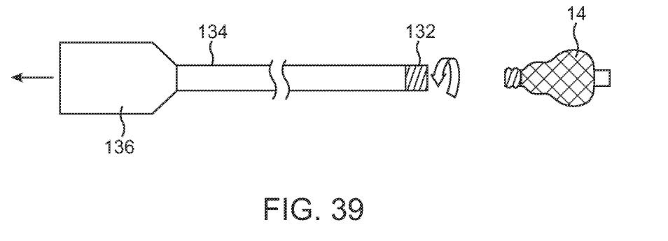

FIG. 36 illustrates a screw attachment delivery system. FIG. 36 shows the delivery system with the stent 14 fully unsheathed and detached from the system. The attachment mechanism 130 of this embodiment is similar to that of a machine screw and machine nut. The proximal end of the stent 14 has external threads that mate with internal threads 132 inside the control shaft 134 of the delivery system. Similarly, the external threads can be on the control rod with the internal threads are on the proximal end of the stent. Once the stent 14 is unsheathed and expanded, the control shaft can detach from the stent with a rotational maneuver that unscrews the delivery system off of the stent. FIG. 37 illustrates the screw attachment embodiment of this invention with the outer sheath 20 housing the collapsed stent 14 which is attached to the control shaft 134. The attachment of the control shaft 134 and the stent is accomplished with a screw type of mechanism. The control shaft provides the attachment mechanism to the stent but also provides the necessary column strength to resist the frictional unsheathing forces to follow. FIG. 38 illustrates the screw attachment embodiment of this invention with the stent 14 fully unsheathed but still attached to the control shaft 134. The stent 14 was positioned in the target location and the sheath 20 was retracted. The screw attachment of the stent to the control shaft allows the operator the opportunity to evaluate the position of the stent prior to releasing it. The stent 14 is released by rotating handle 136 (FIG. 39).

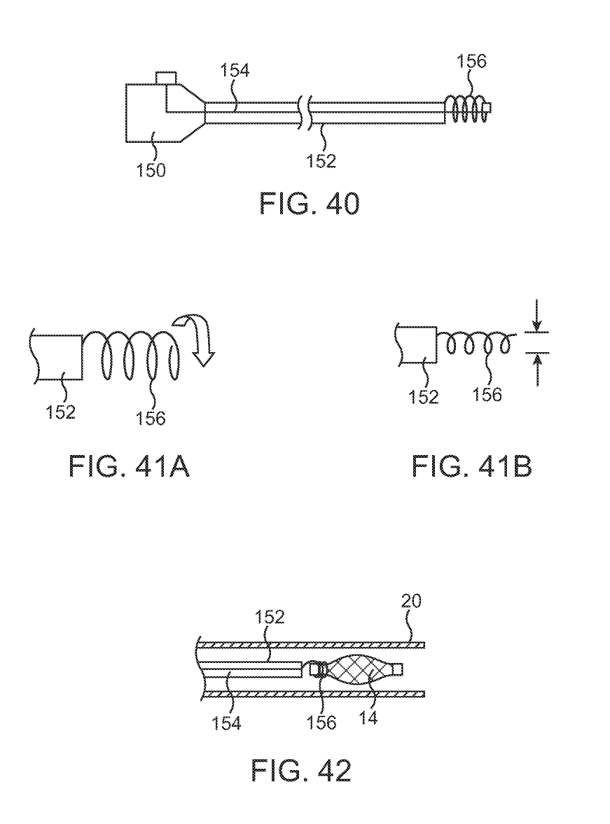

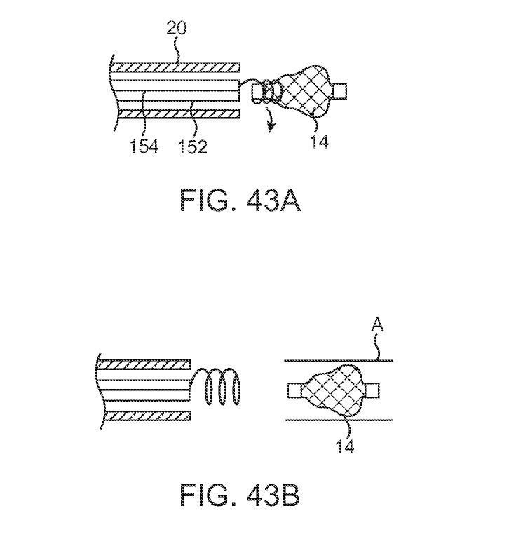

FIG. 40 illustrates a spring snare delivery system comprising a proximal handle 150, a central shaft 152, a control wire 154, and the coil spring lock mechanism 156. The coil spring lock mechanism is what will provide the means of attaching the delivery system to the stent. Locking of the coil spring to the stent will be controlled remotely from the proximal handle through a control wire. As shown in FIGS. 41A and 41B, the coil spring or spring snare 156 reduces its inner diameter with either a torque applied to the spring snare or a tensile load. Either type of loading to the spring snare will reduce its inner diameter. This reduction in diameter provides an interference between the stent and the spring snare which will maintain a hold of the stent. As shown in FIG. 42, the control wire 154 is either maintaining a tension or a torque that reduces the inner diameter of the spring snare. The central shaft 152 provides the necessary column strength to resist the frictional unsheathing forces during the unsheathing operation. As shown in FIGS. 43A and 43B, the stent 14 is fully unsheathed and detached as a tension or torque placed on the spring snare 156 through the control wire 154. The resulting increase in inner diameter allows the stent to be released from the delivery system into airway A. The delivery system is then removed.

It can be appreciated that there are many combinations of the above embodiments that can be combined in different ways for a host of applications. Further it can be appreciated that the interventional catheter can comprise additional features such as features to load an implant into the distal tip of the catheter, steering features, controls for the interventional settings like laser on-off control etc., implant attachment and detachment features, re-sheathing features for when an implant is drawn back into the catheter sheath tip after it is released if so desired, etc. Further, while the embodiments often describe an interventional catheter, that is merely exemplary and other types of interventional instruments are also included in the invention. Further, dimensionally the catheter or instrument assembly described in this invention can comprise a wide range of dimensions, such as larger diameters and lengths used for colon procedures or such as miniature diameters and lengths used for delicate precise inner ear procedures.

What is claimed is:

1. A method for positioning a flow restrictive element in an airway, said method comprising:
advancing the flow restrictive element from a sheath, wherein the flow restrictive element self-expands within the sheath; and
releasing the flow restrictive element within the airway; or
drawing the flow restrictive element back into the sheath.

2. A method as in claim 1, wherein advancing comprises moving a grasping tool which releasably holds the flow restrictive element through a lumen of the sheath and releasing comprises disengaging an attachment member on the grasping tool.

3. A method as in claim 2, further comprising re-engaging the attachment member with the flow restrictive element.

4. A method as in claim 3, further comprising moving the grasping tool to draw the engagement member and flow restrictive element back into the sheath.

5. A method as in claim 3, further comprising moving the grasping tool to reposition the flow restrictive element in the airway.

6. A method as in claim 5, further comprising re-releasing the flow restrictive element in the airway.

7. A method as in claim 1, further comprising positioning the flow restrictive element after said element has been advanced and before said element has been released or drawn back into the sheath.

8. A method as in claim 1, wherein the airway leads to a diseased lung segment.

9. A method as in claim 8, wherein the flow restrictive element is released to occlude air flow into the diseased lung segment.

10. A system comprising:
a self-expanding flow restrictive element adapted for releasing to occlude an airway in a lung;
a delivery sheath having at least one lumen adapted to receive and constrain the self-expanding flow restrictive element; and
a grasping tool having an attachment member which releasably holds the flow restrictive element;
wherein the grasping tool is axially advanced through the delivery sheath lumen to position the flow restrictive element in the airway and the attachment member is actuated to release the flow restrictive member at the location where it has been positioned.
